(19) United States
(12) Patent Application Publication
(10) Pub. No.: US 2006/0200160 A1
(43) Pub. Date: Sep. 7, 2006

(54) Internal fixation assemblies and associated instruments

(75) Inventors: Robert Border, Bourbon, IN (US); Philip Frank, Maplewood, NJ (US); Timothy Elghazaly, Piscataway, NJ (US); Kenneth J. Koval, Hannover, NH (US); Frederick J. Kummer, Brooklyn, NY (US)

Correspondence Address: HARNESS, DICKEY & PIERCE, P.L.C., P.O. BOX 828, BLOOMFIELD HILLS, MI, 48303, US

(73) Assignee: EBI, L.P.

(21) Appl. No.: 11/258,350

(22) Filed: Oct. 25, 2005

Related U.S. Application Data
(60) Provisional application No. 60/654,850, filed on Feb. 18, 2005.
(60) Provisional application No. 60/654,859, filed on Feb. 18, 2005.

(51) Int. Cl. A61B 17/58

(52) U.S. Cl. 606/88

(57) ABSTRACT

An internal fixation assembly and associated method. The internal fixation assembly includes an elongated intramedullary implant for a long bone, the intramedullary implant defining a transverse bore having an inlet opening and an outlet opening, and a strength-enhancing lip surrounding at least one of the inlet and outlet openings. The lip includes a continuous curved surface having a continuous slope.

### CROSS-REFERENCE TO RELATED APPLICATIONS

This application claims the benefit of U.S. Provisional Application No. 60/654,850, filed on Feb. 18, 2005 and U.S. Provisional Application No. 60/654,859, filed on Feb. 18, 2005. The disclosures of the above applications are incorporated herein by reference.

### INTRODUCTION

Different nailing systems and associated instruments are known for the fixation of fractures of the femur, such as shaft fractures, subtrochanteric fractures, intertrochanteric fractures, neck fractures and combinations thereof, as well as for reconstruction of the femur following tumor resection or other surgery.

Although the available nailing systems and instruments can be satisfactory for their intended uses, there is still a need for versatile and effective internal fixation assemblies and associated instruments that can be used for internal fixation of long bones.

### SUMMARY

The present teachings provide an internal fixation assembly. In one aspect, the internal fixation assembly includes an elongated intramedullary implant for a long bone, the intramedullary implant defining a transverse bore having an inlet opening and an outlet opening, and a strength-enhancing lip surrounding at least one of the inlet and outlet openings. The lip includes a continuous curved surface having a continuous slope.

In another aspect, the internal fixation assembly includes an elongated intramedullary implant for a long bone, the intramedullary implant defining a transverse bore having an inlet opening and an outlet opening, a transverse member receivable in the transverse bore; an engagement assembly receivable in a proximal axial bore of the intramedullary implant, the engagement assembly comprising a pair of engagement arms for engaging the transverse member and substantially preventing rotation of the transverse member relative to the transverse bore.

The present teachings also provide an internal fixation method. The method includes providing an intramedullary implant defining a proximal axial bore and a transverse bore, the intramedullary implant including an engagement assembly having two engagement arms within the axial bore, inserting the intramedullary implant into a bone, inserting a transverse implant into the transverse bore, and coupling the engagement arms to the transverse implant.

Further areas of applicability of the present invention will become apparent from the detailed description provided hereinafter. It should be understood that the detailed description and specific examples are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is a partial perspective view of a fixation assembly according to the present teachings, the fixation assembly shown implanted in a femur;

FIG. 2A is a perspective view of an intramedullary implant for the fixation assembly of FIG. 1;

FIGS. 2B-2C are enlarged views of portions of the intramedullary implant of FIG. 2A;

FIG. 2D is a side view of a portion of the intramedullary implant of FIG. 2A;

FIG. 2E is an anterior view of an intramedullary implant according to the present teachings;

FIG. 2F is a lateral view of an intramedullary implant according to the present teachings;

FIG. 2G illustrates a staggered arrangement of bores for an intramedullary implant according to the present teachings;

FIG. 2H illustrates an angulated arrangement of bores for an intramedullary implant according to the present teachings;

FIG. 3 is perspective view of a fixation assembly illustrating a coupling detail between intramedullary and transverse implants according to the present teachings;

FIG. 4 is a perspective view of the fixation assembly of FIG. 3, illustrating size variations;

FIG. 5A-5D are perspective views illustrating coupling details for various intramedullary and transverse implants according to the present teachings;

FIGS. 6A-6D are perspective views of various transverse implants for a fixation assembly according to the present teachings;

FIG. 7 is a perspective view of a distal bone screw for a fixation assembly according to the present teachings;

FIGS. 8A-9C illustrate preparation aspects for implanting an intramedullary implant in a femur according to the present teachings;

FIG. 9D is a perspective view of a reamer according to the present teachings;

FIGS. 9E and 9F are perspective views of the components of the reamer of FIG. 9D;

FIG. 10 is an environmental view of a driver assembly according to the present teachings;

FIG. 11 is a enlarged detail of FIG. 10;

FIGS. 12-14 are various perspective views and portions thereof of a targeting assembly according to the present teachings;

FIGS. 15 and 16 illustrate the use of a guide wire with a driver assembly according to the present teachings;

FIGS. 17A-17D are various perspective views and portions thereof of an adjustable reamer assembly according to the present teachings;

FIGS. 18A-18C illustrate aspects of the reaming procedure using the reamer assembly of FIG. 17A;

FIGS. 19A-19C illustrate the use of an inserter with a telescoping lag screw according to the present teachings;

FIGS. 20A-20C illustrate aspects of the use of an inserter with a driver assembly according to the present teachings;

FIGS. 21 and 22 illustrate lag screw compression methods according to the present teachings;

FIGS. 23-25 illustrate the use of an inserter with a non-telescoping lag screw according to the present teachings;

FIG. 26 is a perspective view of an instrument assembly according to the present teachings;

FIG. 27 is a perspective view of the instrument assembly of FIG. 26 shown in use for an interconnecting procedure; and

FIGS. 28 and 29 are perspective views of the instrument assembly of FIG. 26 shown in use for a reconstructive procedure.

### DETAILED DESCRIPTION

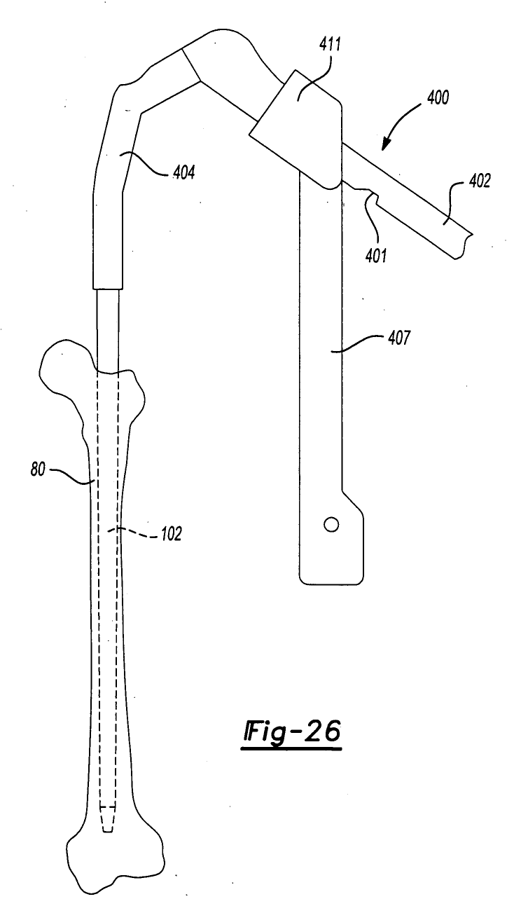

The following description is merely exemplary in nature and is in no way intended to limit the invention, its application, or uses. For example, although the present teachings are illustrated for exemplary trochanteric, reconstructive and interlocking fixation procedures associated with the femur, the present teachings can be used for other fixation procedures involving long bones. In particular FIGS. 1-25 illustrate exemplary trochanteric fixations and associated instruments, and FIGS. 26-29 illustrate a modular instrumentation that can be adapted for use in both interlocking fixation applications and reconstructive fixation applications. It will be understood that general surgical procedures are outlined as needed to illustrate the implants, instruments and methods according to the present teachings, while detailed descriptions of standard and known procedures and instruments are omitted for simplicity.

Referring to FIG. 1, an exemplary fixation assembly 100 according to the present teachings is shown implanted in a femur 80 for a trochanteric application. The fixation assembly 100 includes an elongated intramedullary implant 102 having a shaft 110 received in the intramedullary canal of the femur 80, and an elongated transverse implant 104 coupled transversely to the intramedullary implant 102 and extending from the vicinity of the trochanter 82 and through the neck 86 to the head 84 of the femur 80.

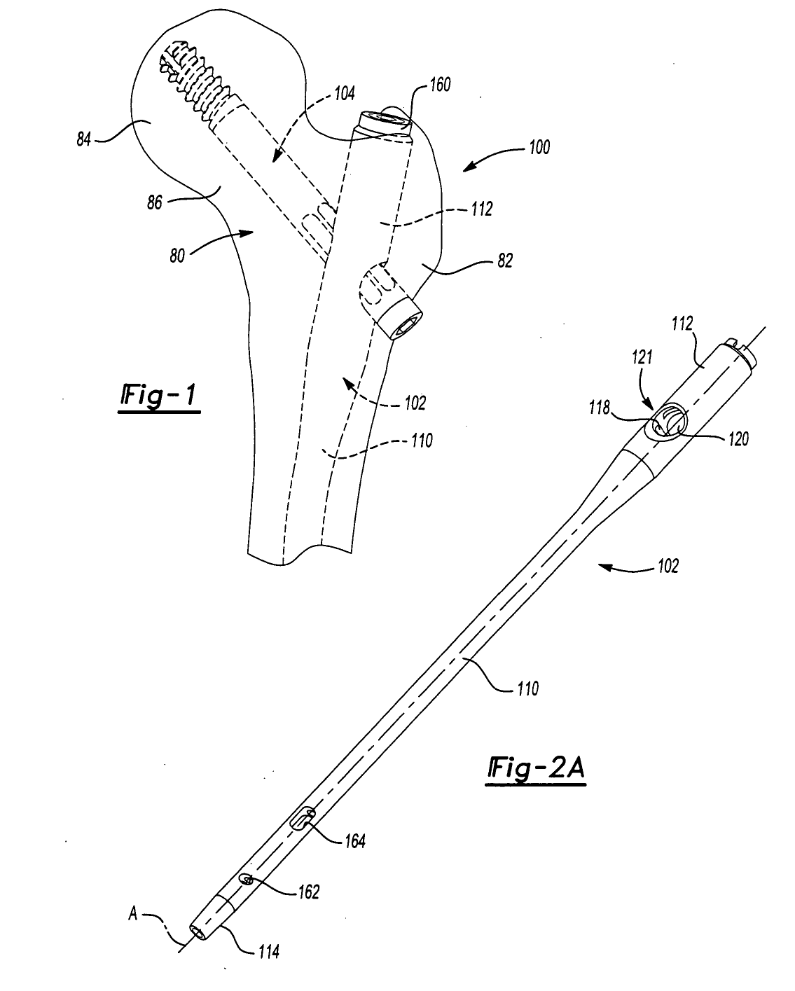

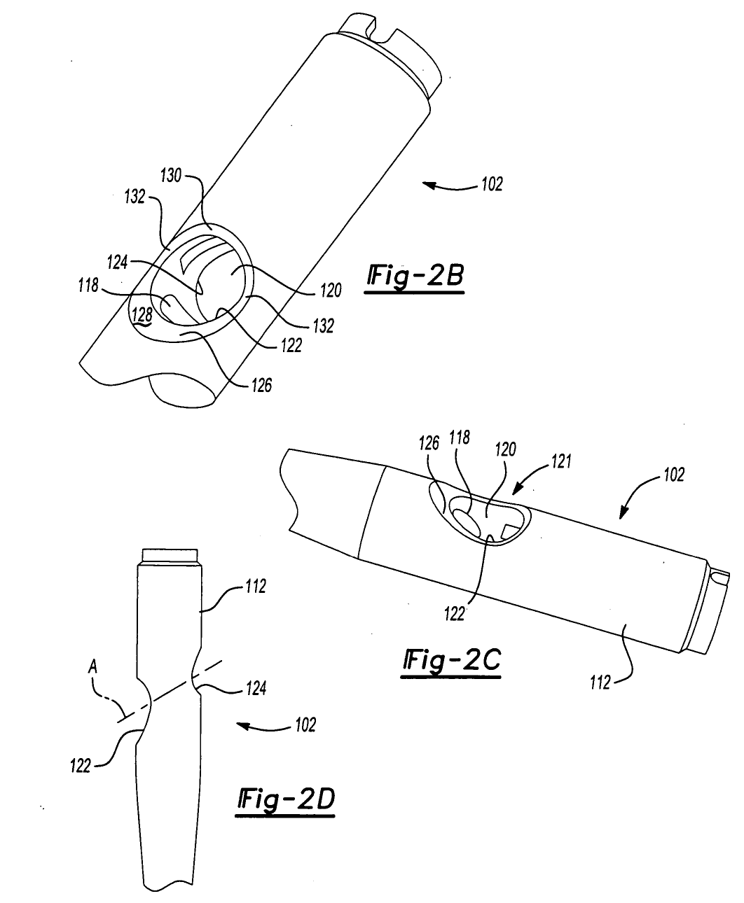

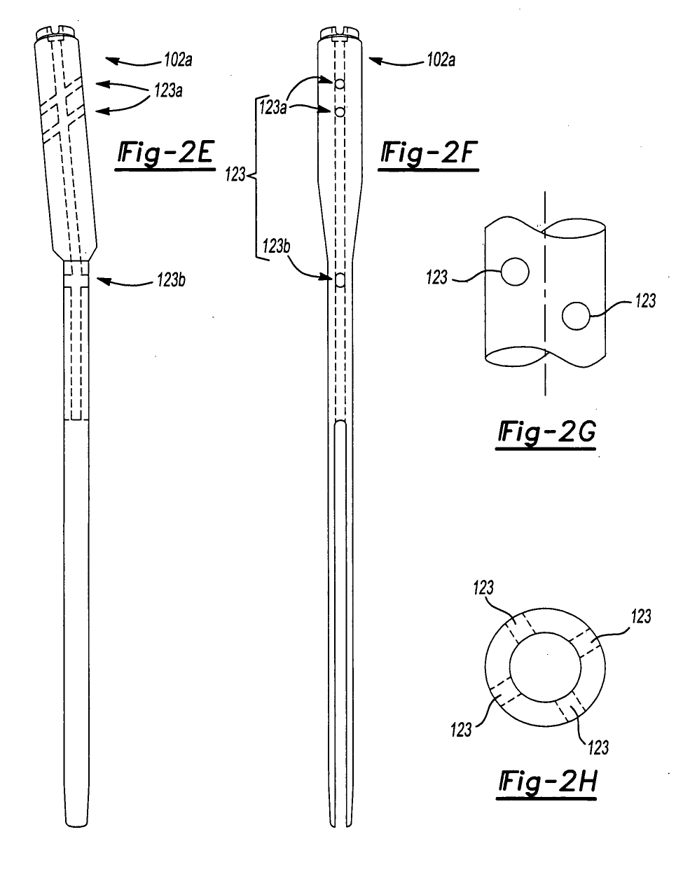

Referring to FIGS. 2A-2D, the intramedullary implant 102 is cannulated defining a longitudinal inner (or intramedullary) bore 118, and includes a proximal portion 112 and a distal portion 114 at corresponding ends of the shaft 110. The proximal portion 112 includes a transverse bore 120 having a central axis A, which is transverse (at an angle) relative to the proximal portion 112, for receiving the transverse implant 104 therethrough. The transverse bore 120 includes an inlet opening 122 and an outlet opening 124. At the inlet opening 122, and optionally at the outlet opening 124, the transverse bore 120 can define a strength-enhancing feature 121, such as a cut defined by an annular curved surface, which is described in connection with the inlet opening 122, although a similar feature 121 can be used in connection with the outlet opening 124. The inlet and outlet openings 122, 124 can have a contour that can be oval, circular or otherwise curved. The inlet opening 122 can be surrounded by a lip 126 defined by a curved surface that can be configured to eliminate any sharp edges or a sharp transition between the transverse bore 120 and the outer surface of the proximal portion 112. The configuration and contour of the inlet opening 122 and lip 126 cooperate to define the strength-enhancing feature 121, which can provide stress relief at the inlet opening 122, reduce contact stresses on the transverse implant 104 and improve strength. The lip 126 can have a surface that generally follows the contour of the inlet opening 122 and includes a distal portion 128, a proximal portion 130 and side portions 132. The lip 126 can be configured to have a greater width (distance from the lip 126 to the contour of the inlet opening 122) at the distal portion 128 than at the proximal portion 130. The lip 126 can be configured as a single curved surface which is both continuous and has continuous slope, and therefore lacks slope discontinuities, such as kinks. The lip 126 does not include any planar or flattened portions. The outlet opening 124 can also be provided with a similar lip and corresponding strength-enhancing feature, if desired.

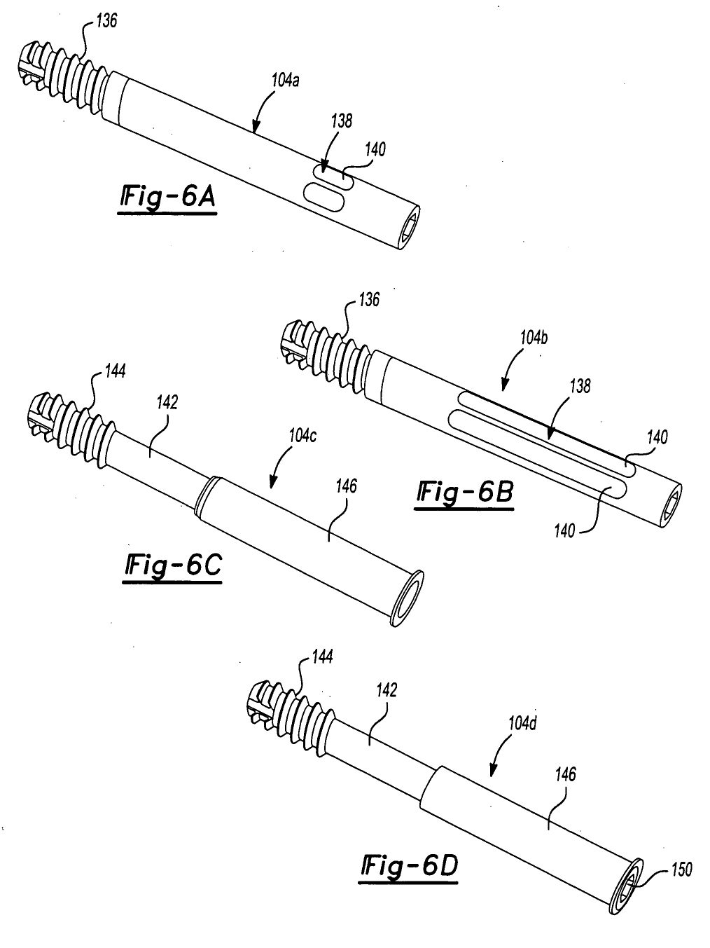

The transverse implant 104 can be constructed to take various aspects appropriate for a variety of different functions and/or applications. Referring to FIGS. 6A-6D, for example, the transverse implant 104 can be, respectively, an integrally formed non-slidable or fixed/static lag screw 104a, an integrally formed slidable or dynamic lag screw 104b, a telescoping keyless lag screw 104c, or a telescoping keyed lag screw 104d. It will be appreciated, however, that other known transverse implants 104 can be used with the fixation assembly 100.

Referring to FIGS. 6A and 6B, each of the fixed and slidable lag screws 104a, 104b can be formed as a single piece including an anchoring end portion 136, which can be, for example, threaded, and a shaft portion 138. The shaft portion 138 of the slidable lag screw 104b includes one or more elongated flat panels 140 for slidable coupling with the intramedullary implant 102, as will be discussed below. In contrast, the shaft portion 138 of the fixed lag screw 104a includes short flat panels 140 that reduce or substantially prevent sliding motion relative to the intramedullary implant 102, as needed in a particular application. For example, the short flat panels 140 can be sized to provide interference against sliding of the fixed lag screw 104a. Conversely, it will be appreciated that any suitable range of sliding within anatomical limits can be provided by selecting an appropriate length for the flat panels 140.

Referring to FIGS. 6C and 6D, each of the telescoping keyless and keyed lag screws 104c, 104d can include a telescoping shaft 142 with a threaded or other anchoring end portion 144, and a tubular sleeve 146 sized to receive the telescoping shaft 142. A bearing length of the shaft 142 is retained within the sleeve 146 and is hidden from the view of FIGS. 6C and 6D. The telescoping keyed lag screw 104d includes a key 150, such as, for example, a hex surface, a projection or other orientation-defining formation, for facilitating the installation of the telescoping keyed lag screw 104d in a desired orientation. The length of the tubular sleeve 146 can be selected to be sufficiently long relative to the length of the shaft 142 such that soft tissue impingement but the shaft is essentially eliminated or negligible. For example, the tubular sleeve 146 can be sized such that the shaft 142 can be entirely received into the sleeve 146, with only the anchoring end portion 144 protruding outside the sleeve 146.

It will be appreciated that other configurations for the intramedullary implant 102 can be used with transverse implants 104. Referring to FIGS. 2E-H, an intramedullary nail 102a can include, for example, a plurality of proximal bores 123, such as angled bores 123a, and/or slots 123b for receiving respectively bone fixation screws and nail-locking anti-rotation screws. The bores 123a and/or the slots 123b can be configured in a staggered arrangement relative to the longitudinal axis of the intramedullary nail 102a, as illustrated in FIG. 2G, or in a cross/angulated arrangement relative to the diameter of the intramedullary nail, as illustrated in FIG. 2H.

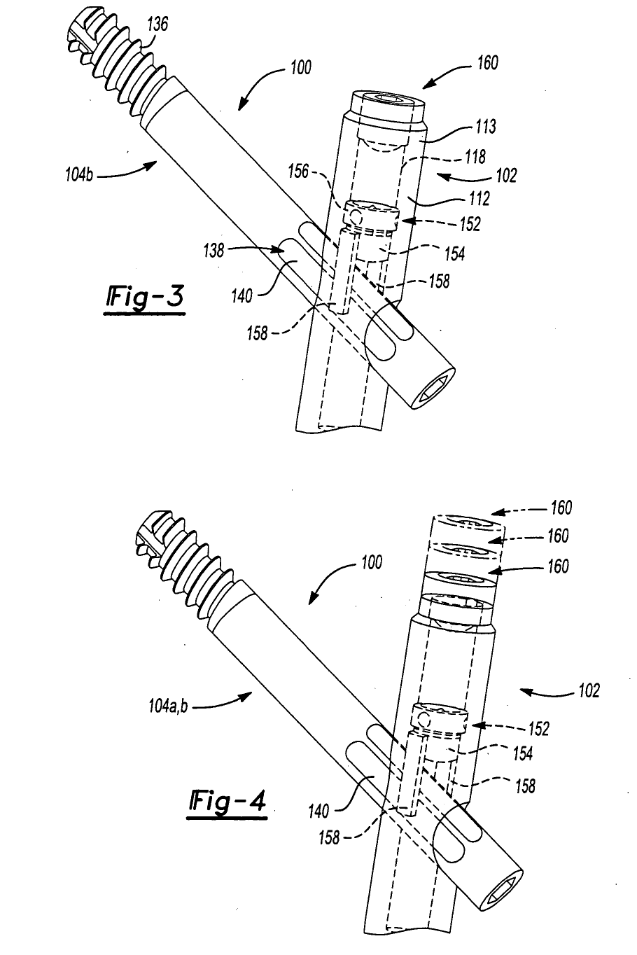

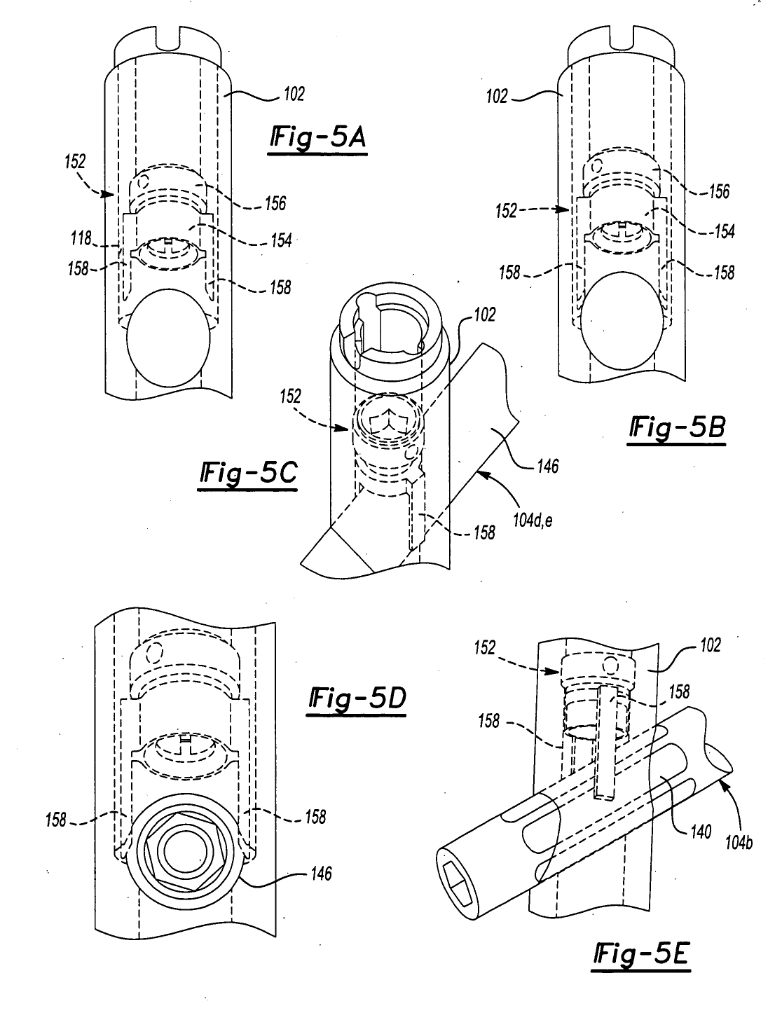

Referring to FIGS. 3, 4, and 5A-5E, exemplary coupling arrangements between the intramedullary and transverse implants 102, 104 are illustrated. A coupling assembly 152 includes an engagement member 154 and an engagement driver 156. The coupling assembly 152 is received in a proximal portion of the intramedullary bore 118 for engaging a portion of the transverse implant 104 that is located within the transverse bore 120. The engagement driver 156 can be threadably coupled with the intramedullary bore 118 and operates to move the engagement member 154 between a disengaged position illustrated in FIG. 5A and an engaged position illustrated in FIG. 5B. The engagement member 154 can include two engagement arms 158 which can engage the sleeve 146 of one of the telescoping lag screws 104c, 104d, as illustrated in FIGS. 5C and 5D, or the flat panels of one of the non-telescoping lag screws 104a, 104b, when the engagement member 154 is in the engaged position. In particular, clockwise and counterclockwise rotation of the engagement driver 156 moves the engagement member 154 between the engaged and disengaged positions. It will be appreciated that the use of sliding or telescoping lag screws 104b, 104c, 104d allows a controlled amount of motion to accommodate anatomic changes after implantation of the fixation assembly 100.

Referring to FIGS. 2A, 3, 4, and 7 the fixation assembly 100 can also include an end cap 160 that can be received at the proximal end 113 of the proximal portion 112 of the intramedullary implant 102 in the intramedullary bore 118. The fixation assembly 100 can also include other bone screws 170 that are inserted through holes 162 or slots 164 at the distal portion 114 of the intramedullary implant 102 for bone fixation.

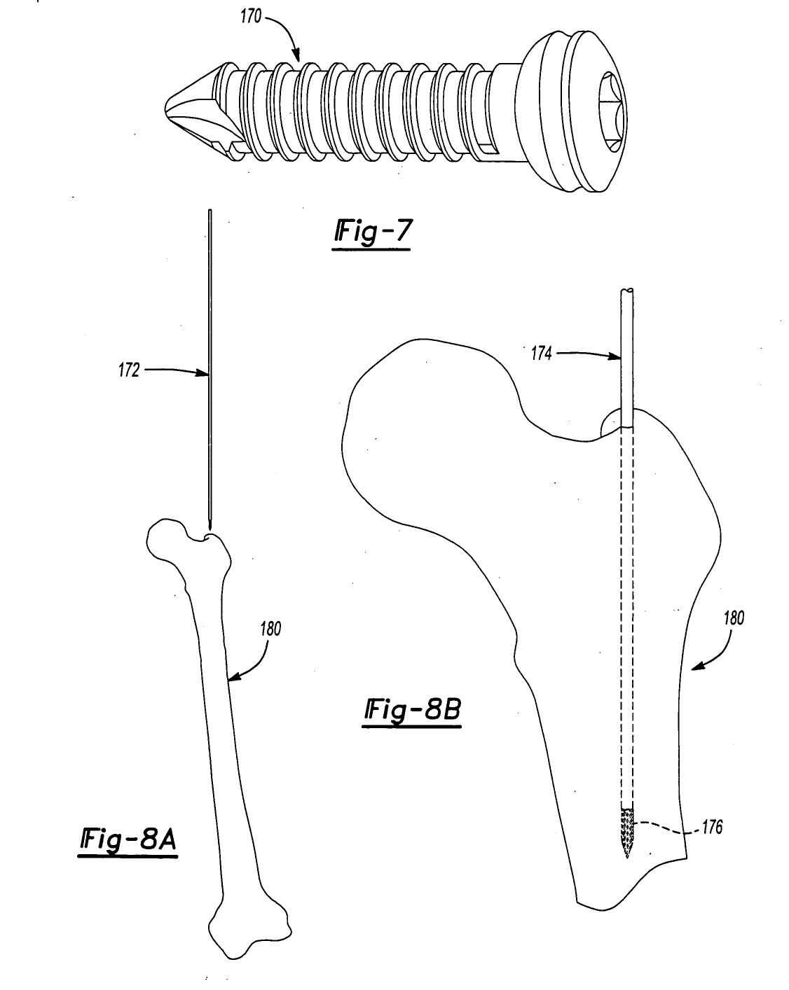

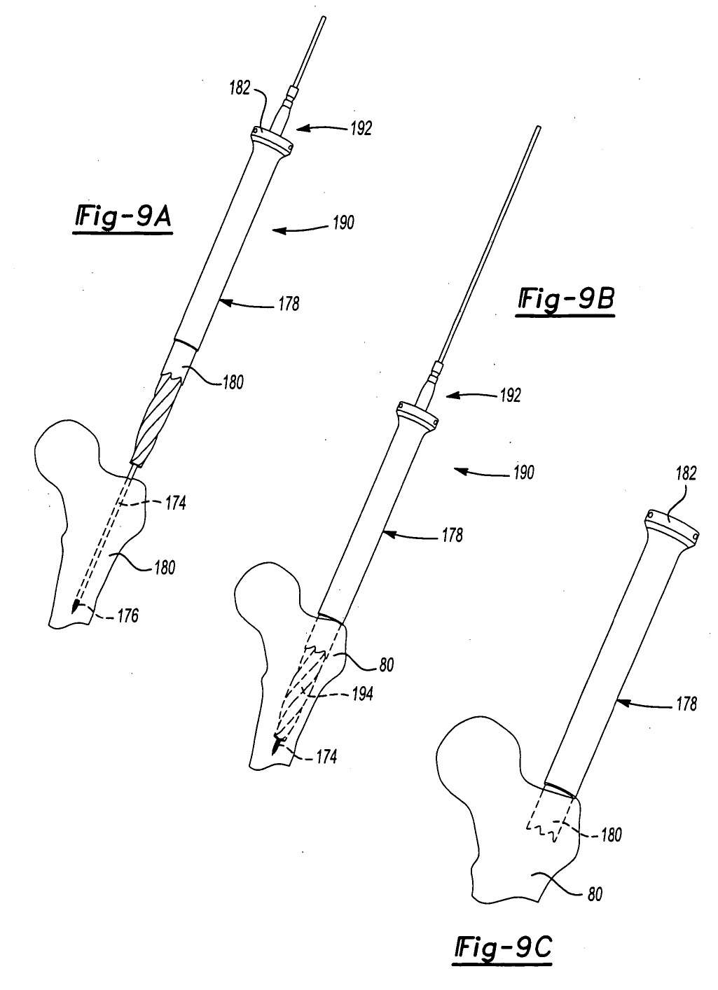

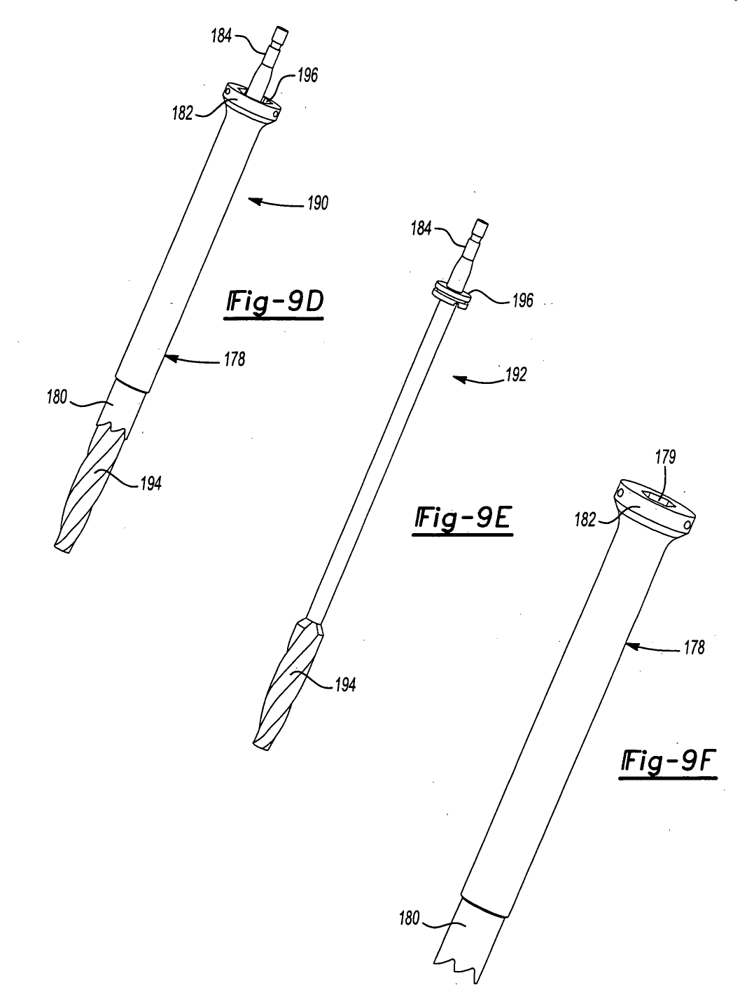

Referring to FIGS. 8-25, an exemplary surgical procedure and associated instrumentation for implantation of the fixation assembly 100 are described next. Referring to FIGS. 8A-9E, the femur 80 is prepared for inserting the intramedullary implant 102. Referring to FIG. 8A, a guide wire 172 can be inserted into the intramedullary canal of the femur 80. Referring to FIG. 8B, an alignment tube 174 is placed over the guide wire 172, the guide wire is removed, and an alignment guide 176 is inserted in the alignment tube 174.

Referring to FIGS. 9A-9F, a one-step proximal reamer assembly 190 and associated reaming procedure is illustrated. The reamer assembly 190 includes a cannula 178 with a longitudinal bore 179 and a reamer 192 that can be received in the bore 179 of the cannula 178 and coupled therein with a quick connect coupling arrangement. The cannula 178 includes a proximal coupling head 182 and a distal edge 180 having teeth. The cannula 178 can be uncoupled from the reamer 192 and can be used separately as a soft tissue sleeve. The reamer 192 is cannulated and has a distal end 184 configured for coupling with a power tool or other driver and a fluted portion 194 for cutting. The reamer 192 can also include a coupling plug 196 having grooves or other engagement formations, such that the plug 196 can be rotated into engagement with mating engagement formations in the coupling head 182 of the cannula 178 for a quick connection. As illustrated in FIGS. 9A-9C, the reamer assembly 190 can be placed over the alignment tube 174. Referring to FIG. 9C, after the alignment guide 176, the alignment tube 174 and the reamer 192 are removed, the cannula 178 can be used for further flexible reaming and canal preparation.

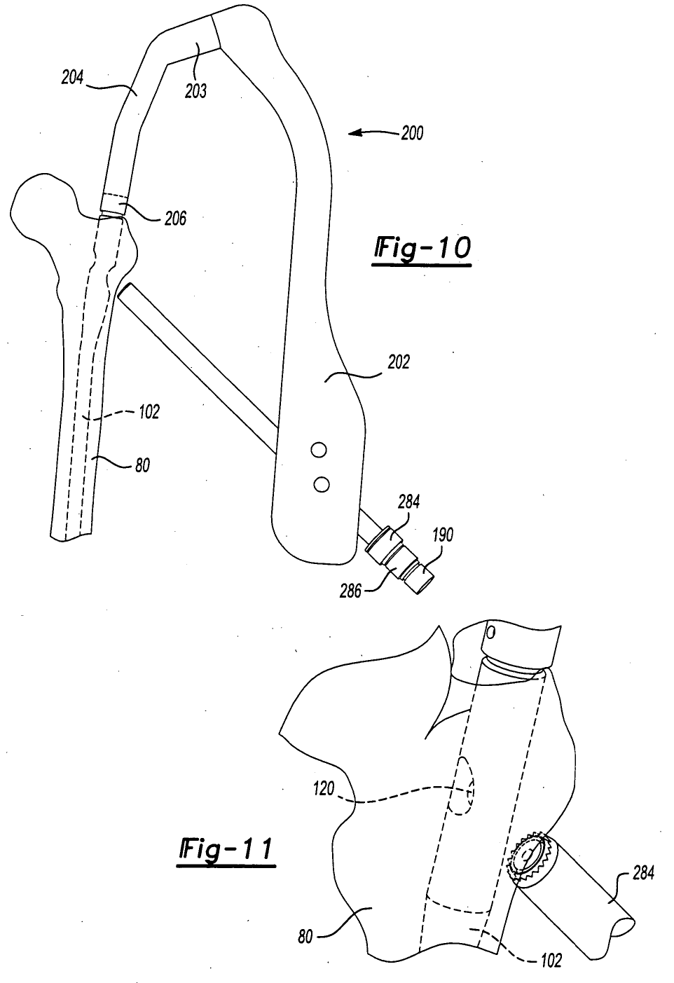

Referring to FIGS. 11 and 10, the intramedullary implant 102 can be first inserted in the prepared canal of the femur 80 using an integrated driver assembly 200, which can also be used for inserting the transverse implant 104 as described below. Alternatively, the intramedullary implant 102 can be implanted using other known procedures and tools.

The driver assembly 200 includes a connector 204, which is typically metallic and is coupled to a radiolucent or carbon driver 202 at one end 203. The other end 206 of the connector 204 is coupled to the proximal end of the intramedullary implant 102. The connector 204 of the driver assembly 200 can be used to implant the intramedullary implant 102 into the femur 80.

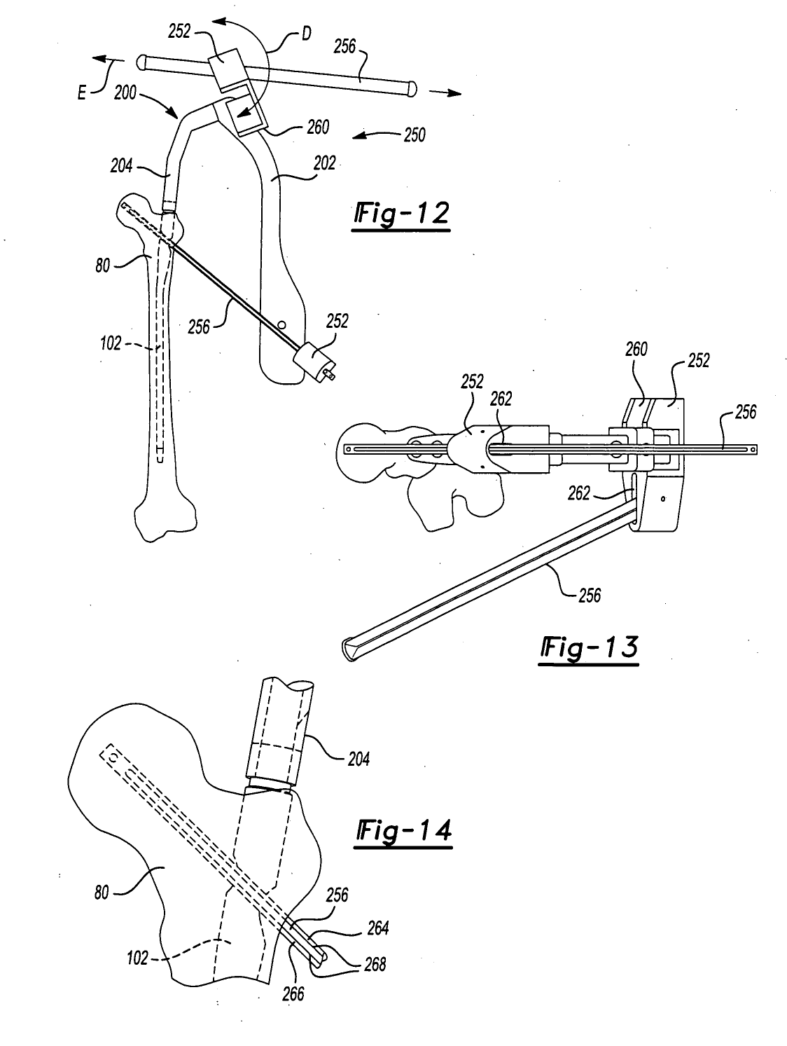

After the intramedullary implant 102 is implanted, the driver assembly 200 can be coupled to a targeting assembly 250, illustrated in FIGS. 12-14, for assisting in the preparation and alignment of a transverse bone canal for receiving the transverse implant 104. The targeting assembly 250 can include a targeting adapter 252 that is configured for coupling to the distal or the proximal end of the driver 202 using appropriately sized clamping jaws or other clamping devices 260 that permit rotation in the direction of a curved arrow D, as shown in FIG. 12. The targeting assembly 250 can also include a targeting outrigger 256 which is slidably received in a slot 262 defined in the targeting adapter 252 and can move in the direction of arrows E. Although two adapters 252 and two outriggers 256 are shown in FIGS. 12 and 13, it will be understood that, if desired, one adapter 252 with an associated outrigger 256 can be used at a time and selectively in one of the two positions illustrated. The outrigger 256 includes a radiolucent housing 264 which receives a radio-opaque bar 266 defining two edges 268. In one aspect, the housing 264 can be made of carbon composite and the radio-opaque bar 266 from metal, although other materials with the desired properties can also be used. In another aspect, the two edges 268 can be defined by two offset metal wires encased in the housing 264 or by a single metal bar machined to present two offset lengths of material.

With continued reference to FIGS. 12 and 13, the targeting assembly 250 can be used to align the driver 202 in the position required for insertion of the transverse implant 104 (as shown in FIG. 10). The position of the targeting outrigger 256 is monitored in a fluoroscope screen (not shown). The targeting outrigger 256 is coupled to the driver 202 consecutively in each of the positions shown and is moved until, in each position, the two edges 268 coincide on the fluoroscope screen. The two positions of the targeting outrigger provide alignment in two non-parallel planes, such that the driver 202 of the driver assembly 200 can be correctly positioned and oriented for reaming and inserting the transverse implant 104 in the desired orientation.

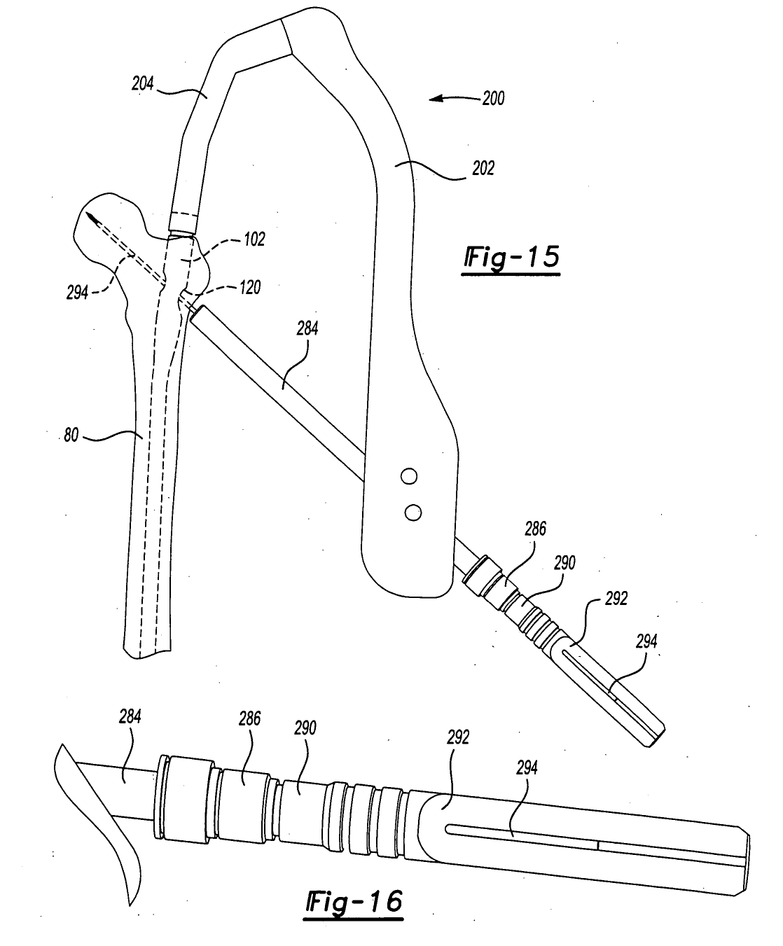

Referring to FIGS. 10 and 15-25, the procedure for implanting the transverse implant 104 is described. As best seen in FIG. 25, the driver 202 includes in its distal portion one or more through apertures 282 for receiving various reaming instruments for inserting the transverse implant 104 through the transverse bore 120 of the intramedullary implant 102. Referring to FIG. 10, the driver 202 is coupled at one of the apertures 282 with a soft tissue sleeve 284, a drill sleeve 286 and a guide wire bushing 290 for preparing and facilitating reaming for the transverse implant 104. Referring to FIGS. 15 and 16, the driver 202 is shown with a guide wire 294 coupled to the guide wire bushing 290 and inserted through the femur 80 and the transverse bore 120. Depth can be measured using a measuring gage 292.

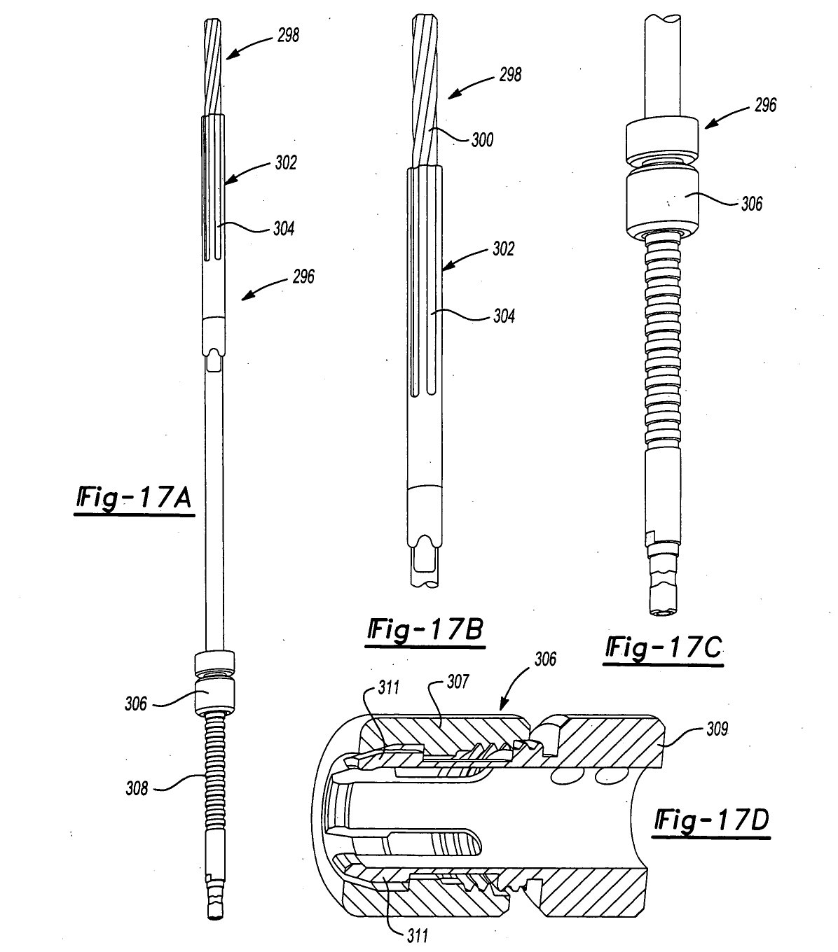

Referring to FIGS. 17A-17D, an adjustable reamer assembly 296 can be used for preparing the transverse bone canal for inserting the transverse implant 104, and in particular one of the telescoping lag screws 104c, 104d. The adjustable reamer assembly 296 can include a first stage cannulated reamer 298 having cutting flutes 300 and a second stage cannulated reamer 302 having cutting flutes 304. The cutting flutes 300, 304 can be spiral or straight or have other known configurations. The second stage reamer 302 forms a sleeve over the first stage reamer 298. The first stage reamer 298 can be uncoupled from the second stage reamer 302, which can be also used as a soft tissue sleeve or working cannula. The length of the reamer assembly 296 can be adjusted to accommodate the length of the transverse implant 104. A stop 306 can be provided to adjust the depth of insertion of the transverse implant 104.

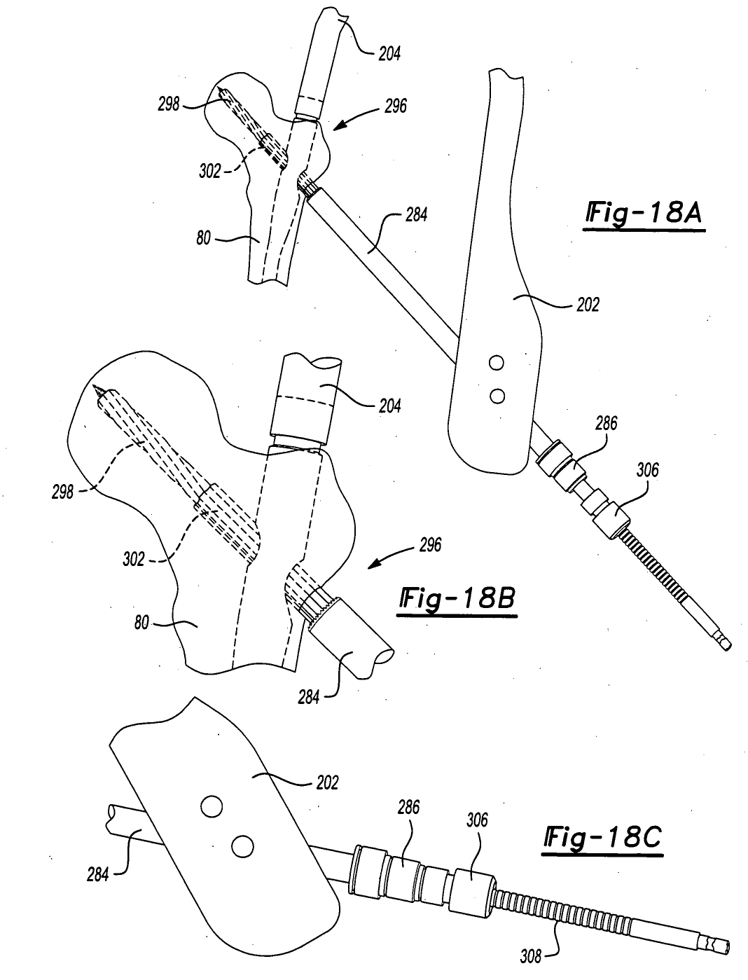

FIGS. 18A-18C illustrate aspects of the reaming procedure using the adjustable reamer assembly 296 through the drill sleeve 286 and soft tissue sleeve 284. In FIG. 18A the stop 306 is spaced away from the drill sleeve 286. In FIG. 18C, the stop 306 abuts the drill sleeve 286 indicating that full depth has been reached. The movement of the stop 306 can be gradated by detent grooves 308 on the reamer assembly 296. Referring to FIG. 17D, the stop 306 can include a first threaded nut 307 that engages a second nut 309. The second nut 309 includes leaf spring tabs 311. Advancing the threaded nut 307 causes compression of the leaf spring tabs 311 and locks the stop 306 into the detent grooves 308.

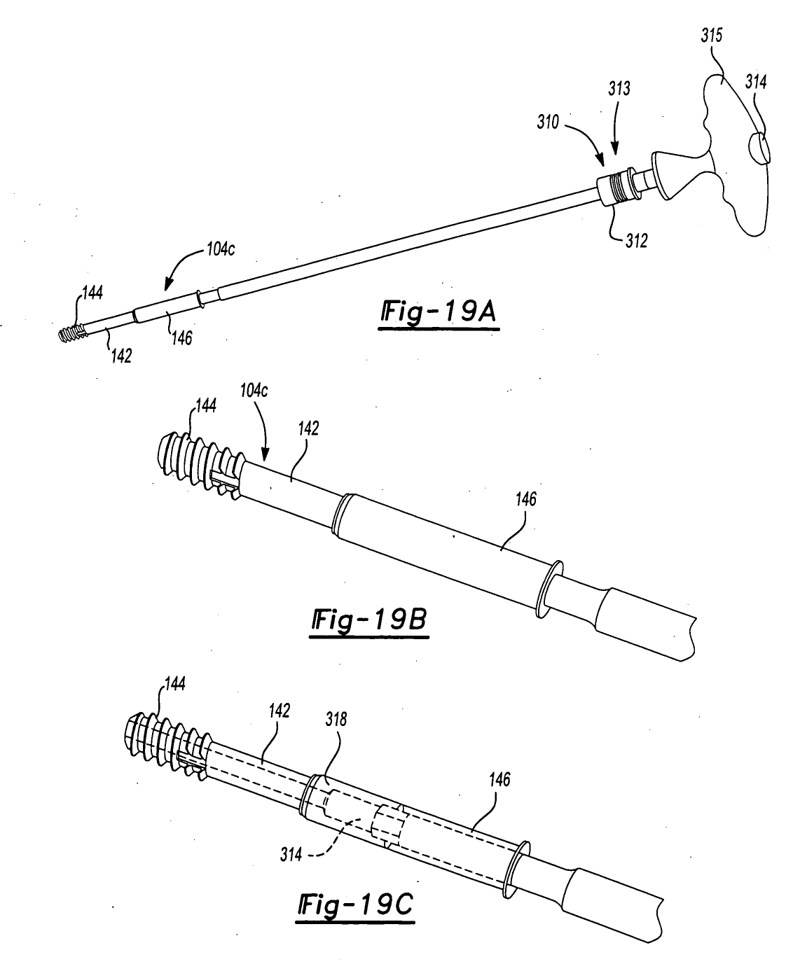

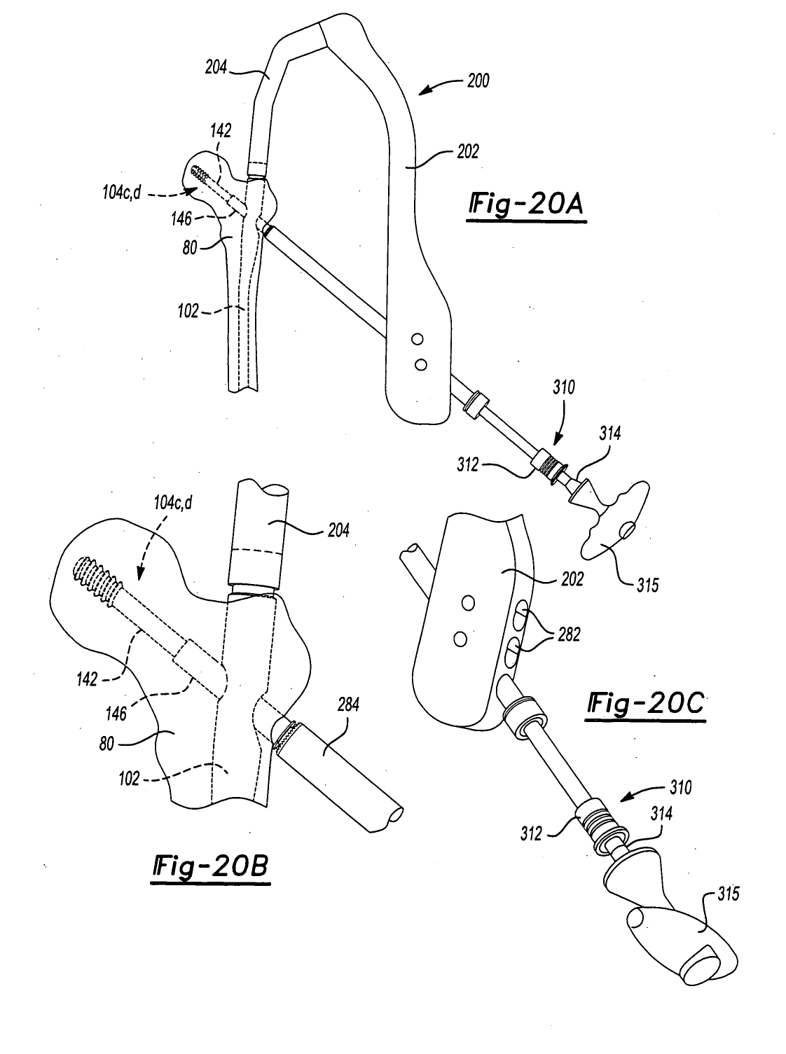

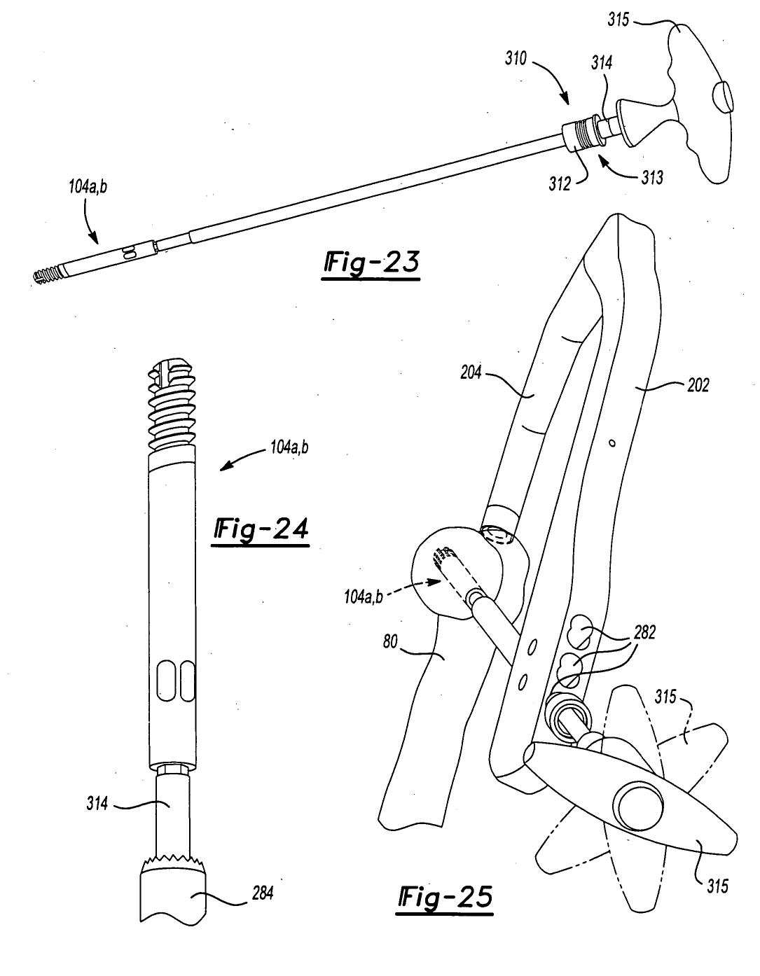

Referring to FIGS. 19A-25 exemplary procedures and devices associated with the insertion of the transverse implant 104 are illustrated. An inserter 310 can be coupled with the transverse implant 104, and in particular, with one of the telescoping lag screws 104c, 104d, as illustrated in FIGS. 19A-19C, or with one of the non-telescoping lag screws 104a, 104b, as illustrated in FIGS. 23-25. The inserter 310 can include a modular quick-connect handle assembly 313 and a threaded cannulated connector shaft 314 to lock the transverse implant 104 to the inserter 310. An engagement connection 318 couples the inserter 310 with the telescoping shaft 142 of the telescoping lag screw 104c, 104d. The handle assembly 313 includes a handle connector 312 and a T-handle 315. Referring to FIGS. 20A-20C, the inserter 310 is shown coupled to the transverse implant 104 through the soft tissue sleeve 284, which is coupled to the driver 202 of the driver assembly 200.

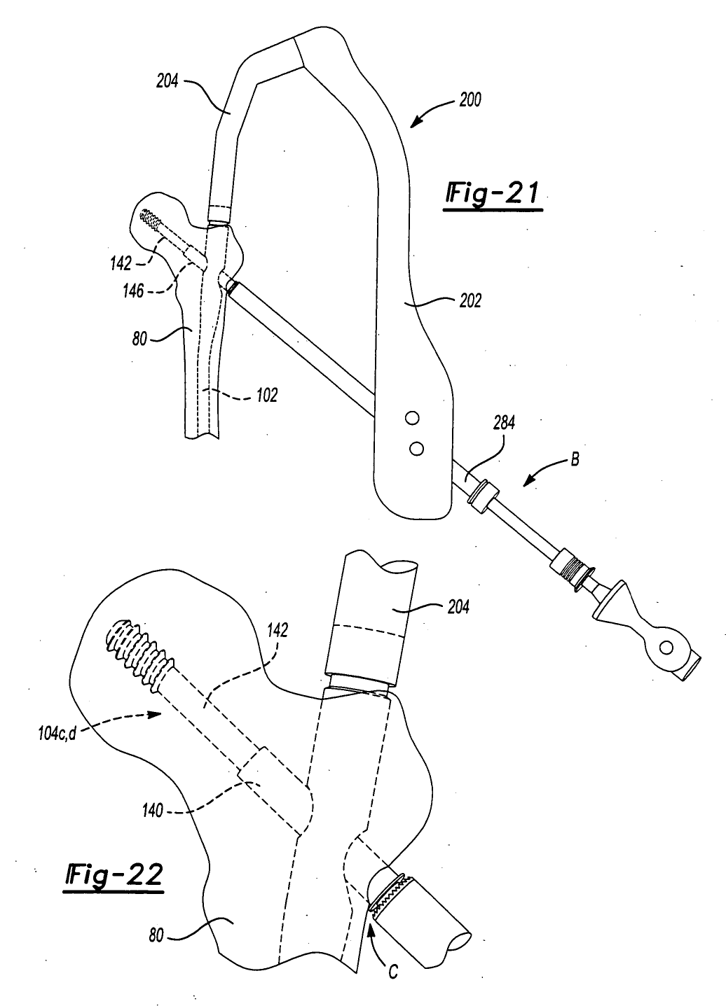

Referring to FIGS. 21 and 22, two exemplary methods of lag screw compression are illustrated. In FIG. 21, compression can be obtained by advancing the connector shaft 314 of the inserter 310 against the soft tissue sleeve 284 after the telescoping shaft 142 of the telescoping lag screw 104c, 104d is in position, as indicated generally at B. Referring to FIG. 22, compression can be obtained by using a shorter telescoping shaft 142 and continue to advance the inserter 310 after the lip of the tubular sleeve 146 of the of the telescoping lag screw 104c, 104d is seated against the lateral cortex, as indicated generally at C.

Referring to FIGS. 23-25, insertion of a non-telescoping lag screw 104a, 104b is illustrated. The connector shaft 314 of the inserter 310 can be coupled to the lag screw 104a, 104b with a hex, square, or other type of engagement connection. The position of the T-handle can be used to indicate the position of the flat panels 140 for alignment with the engagement arms 158 of the coupling assembly 152 (shown in FIG. 3).

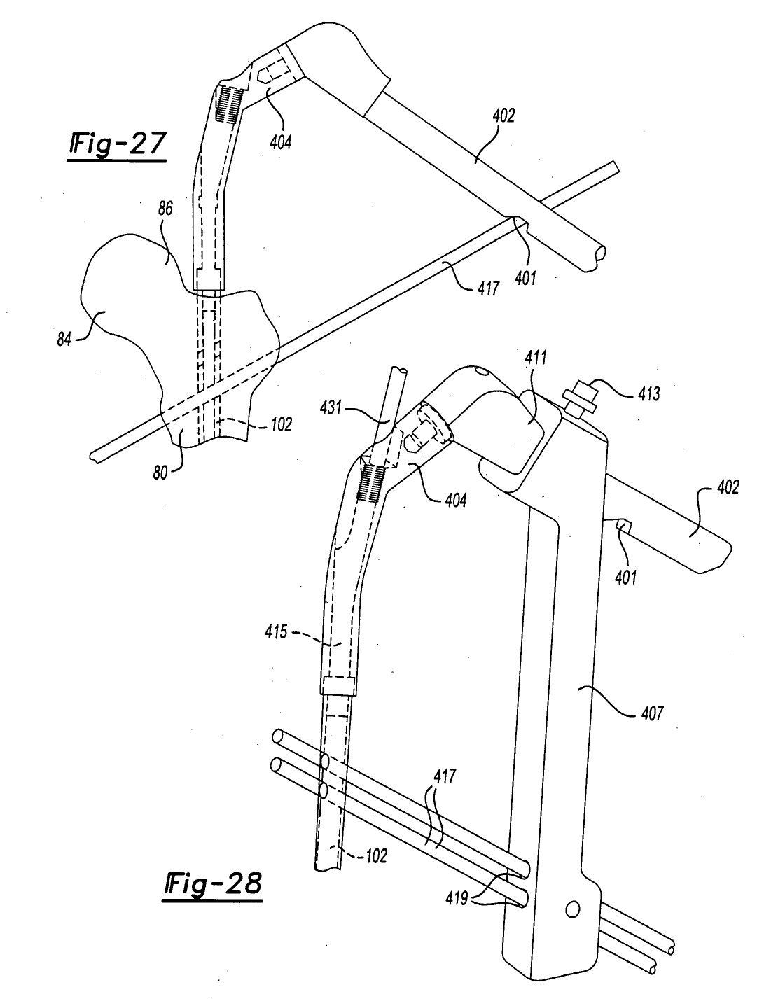

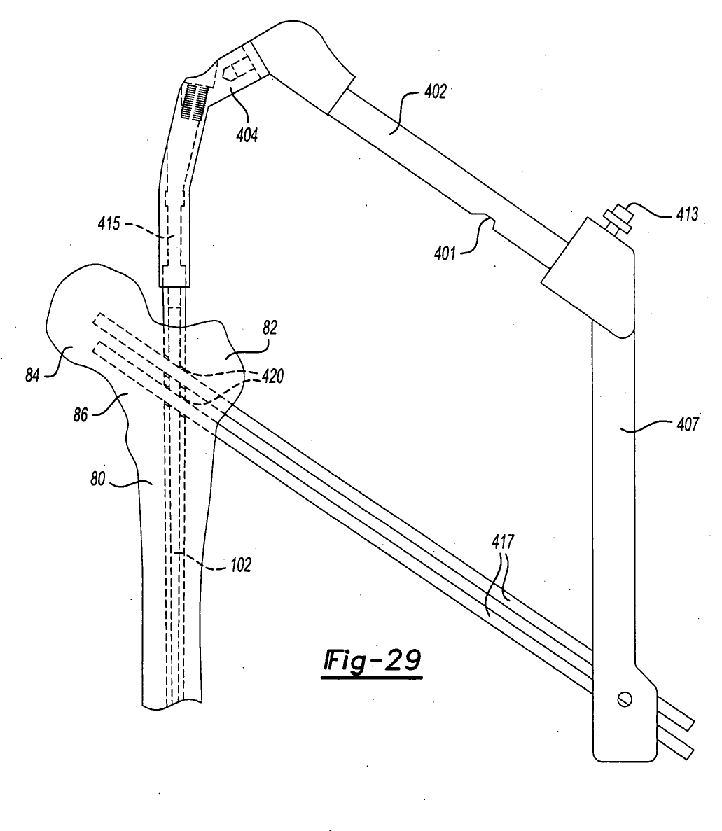

Referring to FIGS. 26-29, a versatile modular instrument assembly 400 that can be used for reconstructive and interlocking indications is illustrated. Reconstructive indications include, but are not limited to, applications in which two proximal screws are inserted from the lateral surface of the trochanter 82 through the femoral neck 86 to the femoral head 84, as illustrated in FIG. 29. Interlocking indications include, but are not limited to applications, in which an interlocking screw is inserted from the lateral to the medial surface of the femur 80 without traversing the femoral head 84, as illustrated in FIG. 27. Features, procedures and instruments that can be common or similar to those described above in connection with the fixation assembly 100 are not described again in connection with the instrument assembly 400. It will be understood, however, that any of the implants and instruments described in connection with FIGS. 1-25 can be used and/or adapted for use with the instrument assembly 400.

The instrument assembly 400 includes a radiolucent driver 402 and a radiolucent or radio-opaque connector 404 coupled to an intramedullary implant 102 through a radio-opaque or metallic connecting bolt 415. The driver 402 can include one or more apertures 401 for attaching a working cannula 417, such as, for example, the cannula 178 or the soft tissue sleeve 284 shown in FIGS. 9C and 11, respectively. The working cannula 417 can be coupled with a corresponding transverse hole 420 in the intramedullary implant 102 for interlocking applications, as illustrated in FIG. 29.

The instrument assembly 400 can include a radiolucent arm 407 that has a proximal end defining an opening 411 for slidably receiving the driver 402. The arm 407 can be secured at any position along the driver 402 using a securing device, such as a set screw 413, as shown in FIGS. 28 and 29. The arm 407 has a distal end with one or more holes 419 for receiving a corresponding working cannula 417, which can be coupled with corresponding transverse holes 420 in the intramedullary implant 102. It will be appreciated that the arm 407 can be removed from the instrument assembly 400 for interlocking applications, such as those illustrated in FIG. 27, thereby eliminating potential compounding of tolerances and simplifying the work of the surgeon in such procedures.

Referring to FIG. 28, the instrument assembly can further include a long, removable driver 431 for locking the connecting bolt 415. A removable boss can be used to impact the intramedullary implant 102 at a surface outside the implant area. A slap hammer can also be connected to apply impact loads in either direction.

The foregoing discussion discloses and describes merely exemplary arrangements of the present invention. One skilled in the art will readily recognize from such discussion, and from the accompanying drawings and claims, that various changes, modifications and variations can be made therein without departing from the spirit and scope of the invention as defined in the following claims.

What is claimed is:

1. An internal fixation assembly comprising:
an elongated intramedullary implant for a long bone, the intramedullary implant defining a transverse bore having an inlet opening and an outlet opening; and
a strength-enhancing lip surrounding at least one of the inlet and outlet openings, the lip comprising a continuous curved surface having a continuous slope.

2. The internal fixation assembly of claim 1, wherein the lip is oval-shaped.

3. The internal fixation assembly of claim 1, further comprising:
a transverse member receivable in the transverse bore; and
an engagement assembly receivable in a proximal axial bore of the intramedullary implant, the engagement assembly comprising a pair of engagement arms for engaging the transverse member and substantially preventing rotation of the transverse member relative to the transverse bore.

4. The internal fixation assembly of claim 3, wherein the transverse member comprises a plurality of flat panels engageable with the pair of engagement arms.

5. The internal fixation assembly of claim 4, wherein the flat panels are sized to allow adjustable sliding of the transverse member relative to the transverse bore.

6. The internal fixation assembly of claim 4, wherein the flat panels are sized to substantially prevent sliding of the transverse member relative to the transverse bore.

7. The internal fixation assembly of claim 3, wherein the transverse member comprises:
a shaft having an anchoring end portion; and
a sleeve for preventing soft-tissue impingement, the sleeve sized for telescopically receiving therein substantially the entire shaft excepting the anchoring end portion.

8. The internal fixation assembly of claim 1, further comprising at least another transverse bore, wherein the transverse bores are configured in a staggered or cross arrangement.

9. An internal fixation assembly comprising:
an elongated intramedullary implant for a long bone, the intramedullary implant defining a transverse bore having an inlet opening and an outlet opening;
a transverse member receivable in the transverse bore; and
an engagement assembly receivable in a proximal axial bore of the intramedullary implant, the engagement assembly comprising a pair of engagement arms for engaging the transverse member and substantially preventing rotation of the transverse member relative to the transverse bore.

10. The fixation assembly of claim 9, further comprising an instrument assembly operable for engaging and disengaging the engagement assembly to and from the transverse member.

11. The fixation assembly of claim 10, further comprising:
a cannulated connector;
a radiolucent driver couplable at an angle to the connector; and
a connecting member receivable in the proximal axial bore for connecting the intramedullary implant to the cannulated connector.

12. The internal fixation assembly of claim 11, further comprising a radiolucent arm adjustably connectable to the driver, the radiolucent arm defining at least one opening for receiving a cannula oriented toward the transverse bore.

13. The internal fixation assembly of claim 11, further comprising a targeting assembly coupled to the connector for aligning the transverse implant.

14. The internal fixation assembly of claim 11, further comprising an adjustable reamer assembly coupled to the driver at an orientation for preparing the transverse implant's insertion.

15. The internal fixation assembly of claim 9, further comprising a strength-enhancing lip surrounding at least one of the inlet and outlet openings, the lip comprising a continuous curved surface having a continuous slope.

16. The internal fixation assembly of claim 9, wherein the transverse member is telescopically adjustable relative to the transverse bore.

17. The internal fixation assembly of claim 9, wherein the transverse member comprises a plurality of flat panels for adjustable slidable engagement with the engagement arms.

18. An internal bone fixation method comprising:
providing an intramedullary implant defining a proximal axial bore and a transverse bore, the intramedullary implant including an engagement assembly having two engagement arms within the axial bore;
inserting the intramedullary implant into a bone;
inserting a transverse implant into the transverse bore;
coupling the engagement arms to the transverse implant.

19. The method of claim 18, wherein coupling the engagement arms to the transverse implant comprises coupling the engagement arms to corresponding flat panels of the transverse implant.

20. The method of claim 18, wherein coupling the engagement arms to the transverse implant comprises:
preventing rotation of the transverse implant; and
preventing sliding of the transverse implant relative to the intramedullary implant.

21. The method of claim 18, wherein coupling the engagement arms to the transverse implant comprises:
preventing rotation of the transverse implant; and
allowing adjustable sliding of the transverse implant relative to the intramedullary implant.

22. The method of claim 18, wherein coupling the engagement arms to the transverse implant comprises:
coupling the engagement arms to a sleeve receiving a telescoping shaft of the transverse implant.

23. The method of claim 18, wherein inserting a transverse implant into the transverse bore comprises preventing soft tissue impingement by the transverse implant.

24. The method of claim 23, wherein preventing soft tissue impingement by the transverse implant comprises retracting a telescoping shaft of the transverse implant into a sleeve of the transverse implant.
